(19) United States
(12) Patent Application Publication
(10) Pub. No.: US 2014/0084522 A1
(43) Pub. Date: Mar. 27, 2014

(54) SURGICAL NEEDLE DRIVER AND METHOD OF MAKING THE SAME

(71) Applicant: Greene Manufacturing Technologies, LLC, dba Britt Manufacturing, Port Huron, MI (US)

(72) Inventor: Clinton D. Torgerson, Port Huron, MI (US)

(73) Assignee: Greene Manufacturing Technologies, LLC, dba Britt Manufacturing, Port Huron, MI (US)

(21) Appl. No.: 13/956,098

(22) Filed: Jul. 31, 2013

Related U.S. Application Data
(62) Division of application No. 12/721,011, filed on Mar. 10, 2010, now abandoned.

(51) Int. Cl. A61B 19/00

(52) U.S. Cl. CPC A61B 19/22
USPC 264/645

(57) ABSTRACT

A surgical needle driver including a pair of jaws. The pair of jaws includes an upper jaw and a lower jaw, where the lower jaw has an outer surface facing an outer surface of the upper jaw. The outer surface of each jaw each has a plurality of polygonal pyramidal teeth configured to engage a surgical needle. Each of the plurality of polygonal pyramidal teeth has more than four exposed faces. A method of manufacturing the needle driver or components of the needle driver includes a powder injection molding process.

### CROSS-REFERENCE TO RELATED APPLICATION

This application is a divisional application of U.S. application Ser. No. 12/721,011, filed on Mar. 10, 2010, the entire contents of which are hereby expressly incorporated by reference.

### BACKGROUND

This invention relates to a surgical tool with a grasping means that is suitable for use in surgeries to grasp needles, tissue, and other instruments.

Surgeons frequently use surgical needles to suture wounds or incisions. Since surgical needles are often small and difficult to manipulate, surgeons commonly employ specially adapted devices called needle drivers. Use of a needle driver gives the surgeon a more secure grip on the needle, better visibility of the needle, and better leverage.

One feature of a needle driver is the ability to securely grip the needle in a certain position to minimize needle misplacement during the suturing procedure. Typical needle drivers have a serrated surface defining a plurality of teeth on the face of the jaw that help grip a surgical needle during the suturing procedure. However, these teeth are normally shaped such that the needle may be securely held in only one or two orientations with respect to the jaws of the needle driver. This places a severe limitation on the dexterity and maneuverability of the needle and needle driver, especially in confined surgical circumstances when the needle driver has a restricted range of motion, for example endoscopic surgery.

### SUMMARY

One embodiment of the present invention is directed toward a needle driver having polygonal pyramidal teeth that allows a surgeon or a surgical robot to precisely handle a surgical needle without a gross repositioning of the needle driver after each successive suture.

In another embodiment of the present invention, a surgical needle driver includes a pair of jaws. The pair of jaws includes an upper jaw and a lower jaw, where the lower jaw has an outer surface facing an outer surface of the upper jaw. The outer surface of at least one jaw has a plurality of polygonal pyramidal teeth configured to engage a surgical needle. Preferably, each of the plurality of polygonal pyramidal teeth has more than four exposed faces. For example, the plurality of polygonal pyramidal teeth may have eight exposed faces. The exposed faces may be planar. Alternatively, the exposed faces may be non-planar, for example, the plurality of polygonal pyramidal teeth may have concave exposed faces. Each of the plurality of polygonal pyramidal teeth may have a pointed apex. In another embodiment, each of the plurality of polygonal pyramidal teeth may have a rounded or flattened apex.

The plurality of polygonal pyramidal teeth may be made of a suitably strong and hard material.

At least one of the upper jaw and the lower jaw of the surgical needle driver may be formed of two or more components, including a holder portion with an inner surface and an insert portion with an outer surface and an inner surface. The plurality of polygonal pyramidal teeth are positioned on the outer surface of the insert portion. The inner surface of the insert portion is affixed to the inner surface of the holder portion. In an alternative embodiment, at least one of the upper jaw and the lower jaw is a one-piece jaw.

The surgical needle driver may have a pair of jaws, where the plurality of polygonal pyramidal teeth on the upper jaw is configured to engage the plurality of polygonal pyramidal teeth on the lower jaw such that an apex of each of the plurality of polygonal pyramidal teeth on the upper jaw fits substantially into a valley created by several adjacent teeth of the plurality of polygonal pyramidal teeth on the lower jaw.

In addition, a method for manufacturing a surgical needle driver with a plurality of polygonal pyramidal teeth may include a powder injection molding process. This method may include preparing a mold for at least one surgical needle driver component such as a jaw, a jaw holder portion, a jaw insert portion having a plurality of polygonal pyramidal teeth, where each of the plurality of polygonal pyramidal teeth has more than four faces; injecting the mold with a feed stock containing a powdered metal or ceramic and a polymer binder; sintering the at least one surgical needle driver component at an elevated temperature to form at least one sintered surgical needle driver component; and assembling the at least one sintered surgical needle driver component with the other surgical needle driver components such that the surgical needle driver includes an upper jaw with an outer surface having a plurality of polygonal pyramidal teeth and a lower jaw with an outer surface having a plurality of polygonal pyramidal teeth. The mold may also be for one complete single piece jaw or a single piece leg of the needle driver.

### BRIEF DESCRIPTION OF THE DRAWINGS

Further developments of the invention will stand out from a description of embodiments with reference to the drawings.

FIG. 1 shows a perspective view of an embodiment of the needle driver with open jaws.

FIG. 2 shows a perspective exploded view of the lower jaw of the embodiment shown in FIG. 1.

FIG. 3 shows a perspective view of the needle driver shown in FIG. 1 with closed jaws.

FIG. 4 shows a perspective view of the needle driver shown in FIG. 1 engaging a needle in a diagonal orientation.

FIG. 5 shows a side view of the needle driver shown in FIG. 1 engaging a needle in a transverse orientation.

FIG. 6 shows a side view of the needle driver shown in FIG. 1 engaging a needle in a diagonal orientation.

FIG. 7A shows a perspective view of the lower jaw of the embodiment shown in FIG. 1 engaging a straight needle in a transverse orientation.

FIG. 7B shows a cross-sectional view of the embodiment shown in FIG. 7A engaging a straight needle in a transverse orientation with the upper ends of the teeth removed to show the points of contact between the teeth and the needle.

FIG. 8A shows a perspective view of the lower jaw of the embodiment shown in FIG. 1 engaging a straight needle in a lengthwise orientation.

FIG. 8B shows a cross-sectional view of the embodiment shown in FIG. 8A engaging a straight needle in a lengthwise orientation with the upper ends of the teeth removed to show the points of contact between the teeth and the needle.

FIG. 9A shows a perspective view of the lower jaw of the embodiment shown in FIG. 1 engaging a straight needle in a diagonal orientation.

FIG. 9B shows a cross-sectional view of the embodiment shown in FIG. 9A engaging a straight needle in a diagonal orientation with the upper ends of the teeth removed to show the points of contact between the teeth and the needle.

FIG. 10A shows a perspective view of the lower jaw of the embodiment shown in FIG. 1 engaging a curved needle.

FIG. 10B shows a cross-sectional view of the embodiment shown in FIG. 10A engaging a curved needle with the upper ends of the teeth removed to show the points of contact between the teeth and the needle.

FIG. 11 shows a perspective view of a needle driver with a one-piece jaw engaging a curved needle.

FIG. 12 shows a perspective view of a polygonal pyramidal tooth with planar exposed faces according to an embodiment of the needle driver.

FIG. 13 shows a perspective view of a polygonal pyramidal tooth with concave exposed faces according to an embodiment of the needle driver.

### DETAILED DESCRIPTION

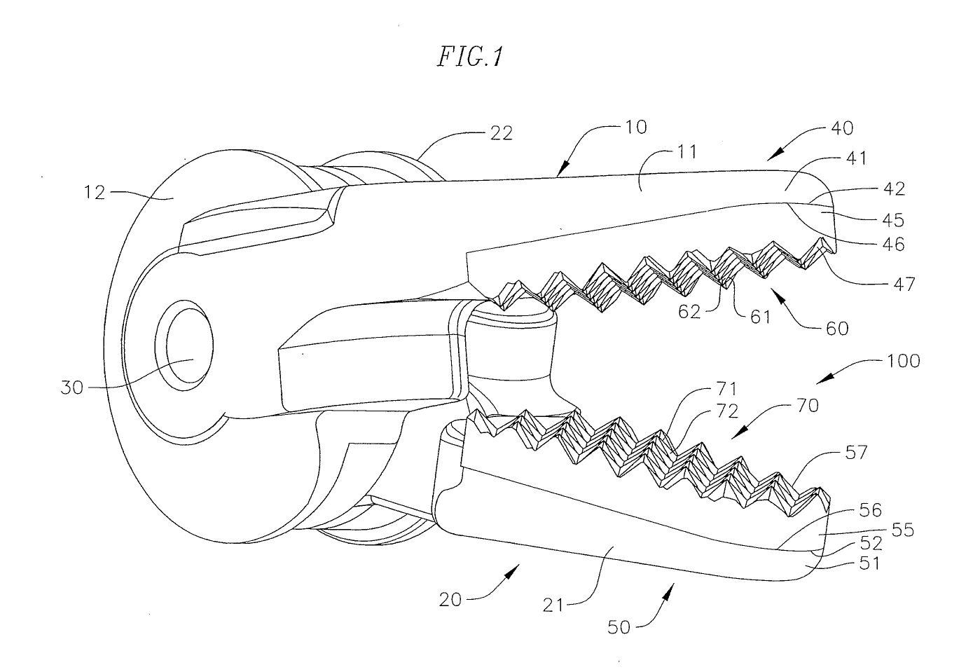

With regard to FIG. 1, an embodiment of a needle driver 100 comprises two legs 10 and 20. Leg 10 has two end portions 11 and 12. Similarly, leg 20 has two end portions 21 and 22. The two legs 10 and 20 are joined to each other by a fulcrum or axle 30 at the respective end portions 12 and 22. On an opposite end of a leg 10 from the fulcrum or axle 30, end portion 11 comprises an upper jaw 40. On an opposite end of a leg 20 from the fulcrum or axle 30, end portion 21 comprises a lower jaw 50. In an alternative embodiment (not shown), the needle driver can be shaped like a pair of pliers or scissors (not shown), as known in the art, that is manually operable by a surgeon.

Movement of the upper jaw 40 and the lower jaw 50 with respect to one another may be actuated by any suitable external device connected with the two legs 10 and 20. Examples of such external devices include, but are not limited to handles, levers, rods, cables, a motor or the like.

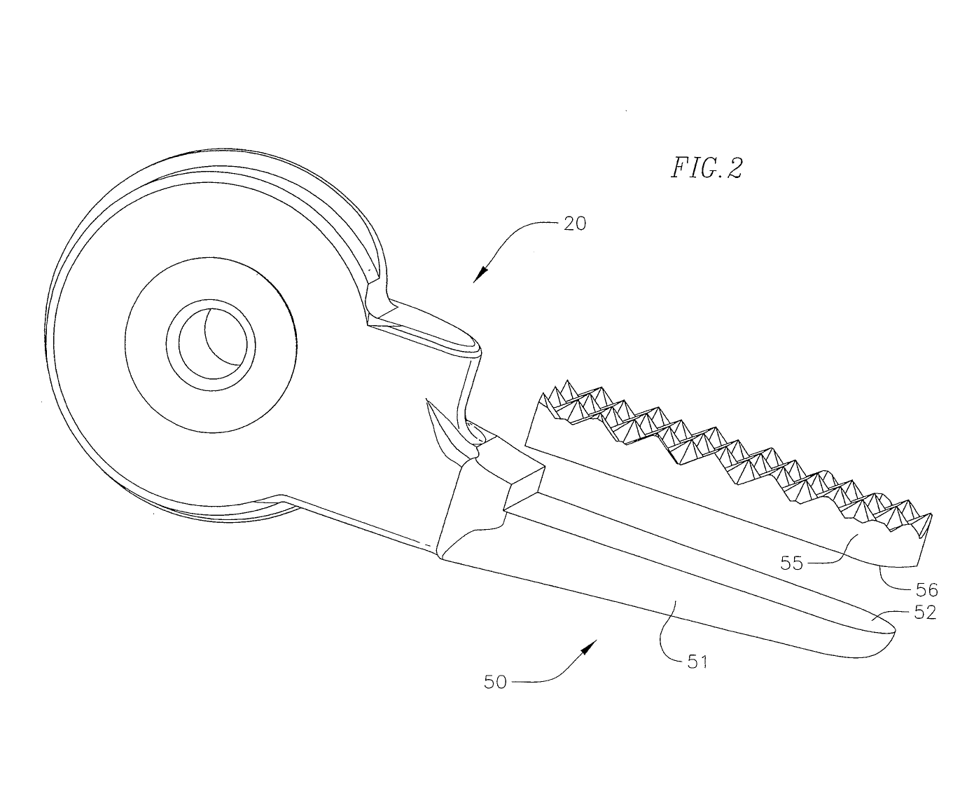

With regard to the needle driver 100 shown in FIG. 1 and the lower leg 20 of the needle driver shown in FIG. 2, the lower jaw 50 includes a holder portion 51 and an insert portion 55. The insert portion 55 has an insert inner surface 56 facing a holder inner surface 52 of the holder portion 51. The insert portion 55 and the holder portion 51 may be joined by any suitable method, such as sinter bonding.

Similarly, the upper jaw 40 includes a holder portion 41 and an insert portion 45. The insert portion 45 has an insert inner surface 46 facing a holder inner surface 42 of the holder portion 41. The insert portion 45 and the holder portion 41 may be joined by any suitable method, such as sinter bonding.

With regard to FIG. 1, the upper insert portion 45 has an outer surface 47 facing an outer surface 57 of the lower insert portion 55. A plurality of polygonal pyramidal teeth 60 are on the upper outer surface 47. Similarly, a plurality of polygonal pyramidal teeth 70 are on the lower outer surface 57.

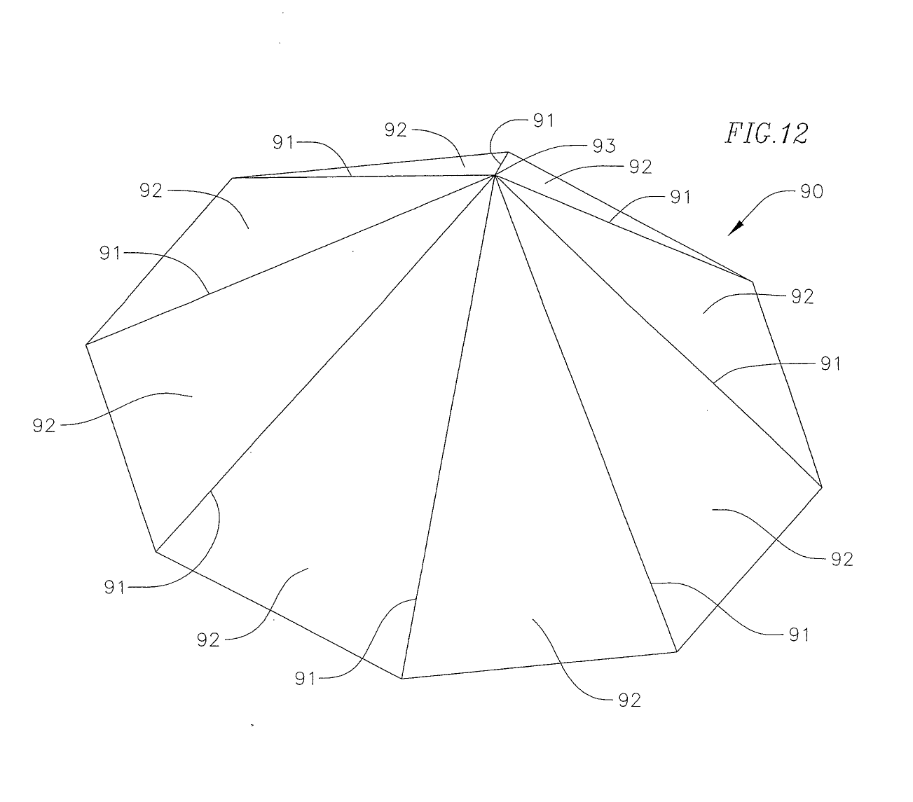

In one embodiment, each tooth of the plurality of lower polygonal pyramidal teeth 70 has eight exposed edges 71 and eight exposed faces 72 (see also FIG. 12). Similarly, each tooth of the plurality of upper polygonal pyramidal teeth 60 has eight exposed edges 61 and eight exposed faces 62. Thus, the pluralities of polygonal pyramidal teeth 60 and 70 in one embodiment comprise pluralities of octagonal pyramidal teeth 60 and 70.

In one embodiment, the insert portion 45 of the upper jaw 40 including the plurality of polygonal pyramidal teeth 60 is formed as a single piece by any suitable method. Examples of suitable methods include machining, casting, molding or powder injection molding. Similarly, the insert portion 55 of the lower jaw 50 may be formed by any of the methods as discussed above in reference to the insert portion 45.

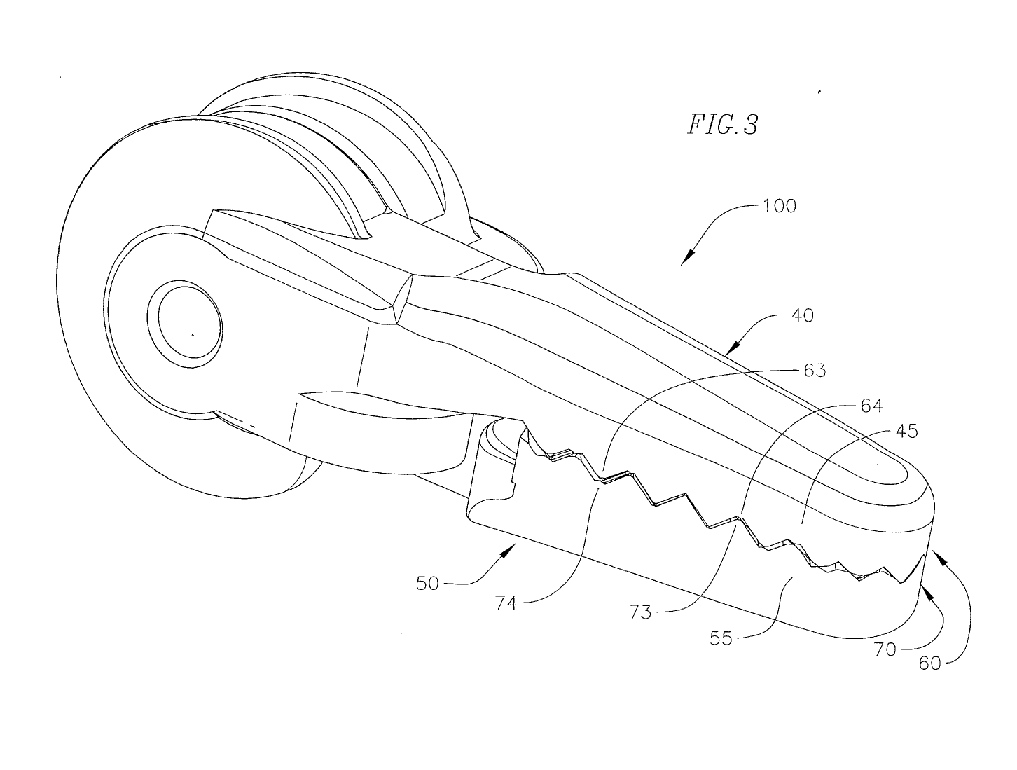

With regard to FIG. 3, the plurality of octagonal pyramidal teeth 60 on the insert portion 45 of the upper jaw 40 are positioned such that an apex 63 of each of the plurality of upper teeth 60 fits substantially into a valley 74 formed by the space between adjacent members of the plurality of lower octagonal pyramidal teeth 70 on the insert portion 55 of the lower jaw 50. Similarly, the plurality of lower octagonal pyramidal teeth 70 on the insert portion 55 are positioned such that a lower apex 73 of each of the plurality of lower octagonal pyramidal teeth 70 fits substantially into an upper valley 64 formed by the space between adjacent members of the plurality of upper octagonal pyramidal teeth 60 on the insert portion 45 of the upper jaw 40. This orientation between the upper and lower jaw allows the teeth to grip the needle. The teeth may also grip the suture thread, for example to tie knots for the suture.

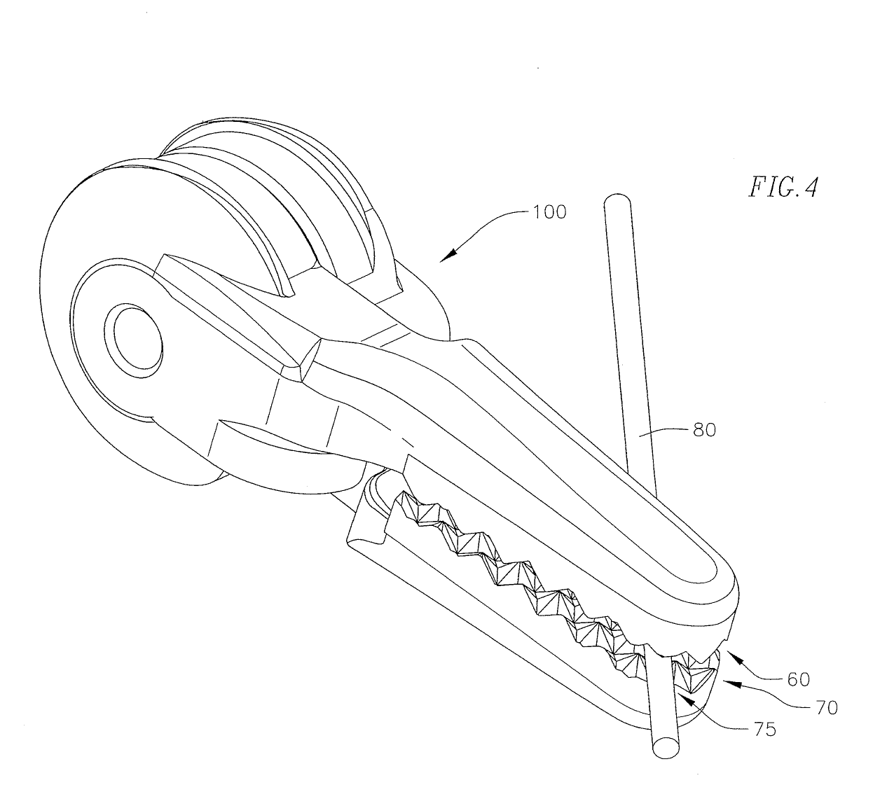

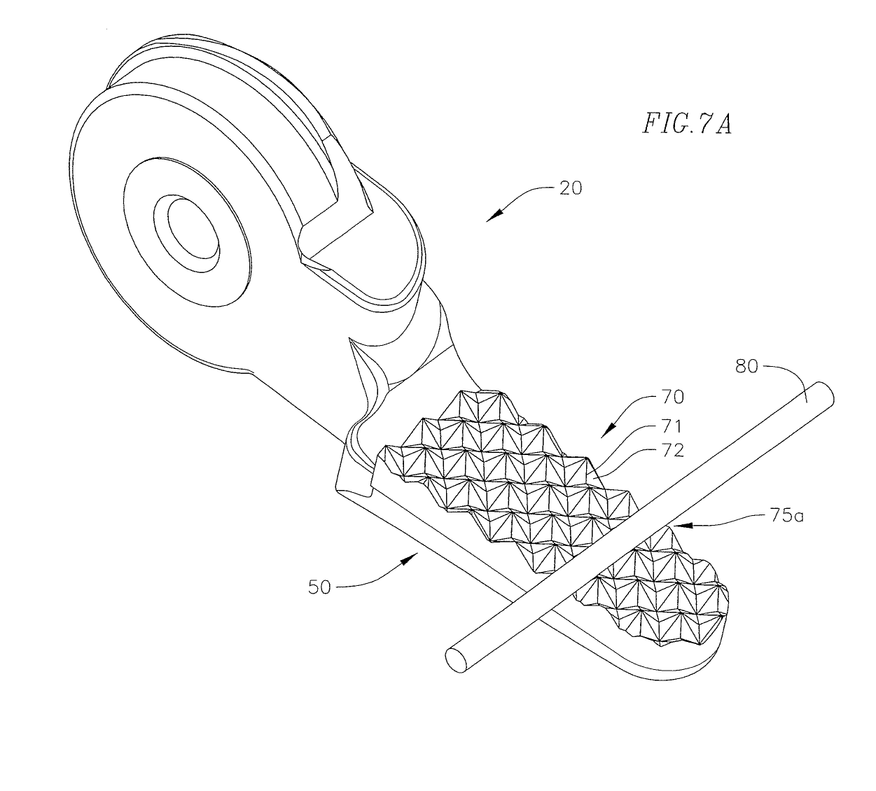

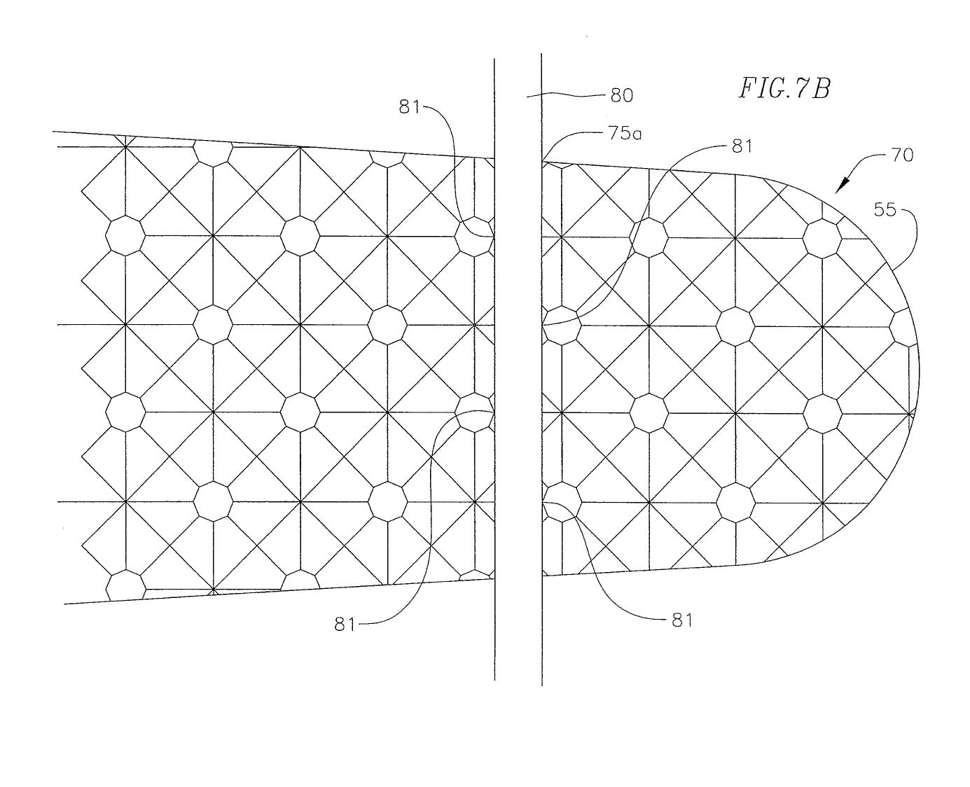

In one embodiment, as shown in FIG. 4, several of the plurality of lower octagonal pyramidal teeth 70 form a lower channel 75. Similarly, several of the upper octagonal teeth 60 form an upper channel (not shown). The upper channel and the lower channel 75 engage a straight needle 80 in an orientation diagonal to the longitudinal axis of the needle driver 100. The needle 80 is engaged by the edges of several of the plurality of octagonal pyramidal teeth 70 (see also FIGS. 7B, 8B, 9B and 10B.

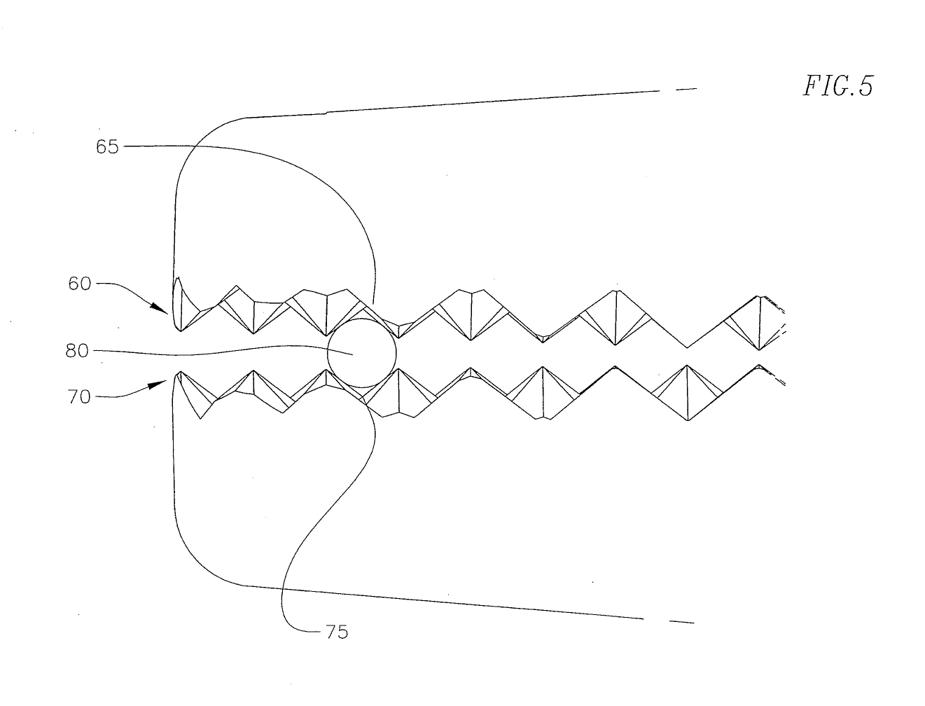

With regard to FIG. 5, several of the plurality of octagonal pyramidal teeth 60 forming an upper channel 65 and several of the plurality of octagonal pyramidal teeth 70 forming a lower channel 75 engage a straight needle 80 in an orientation transverse to the longitudinal axis of the needle driver 100.

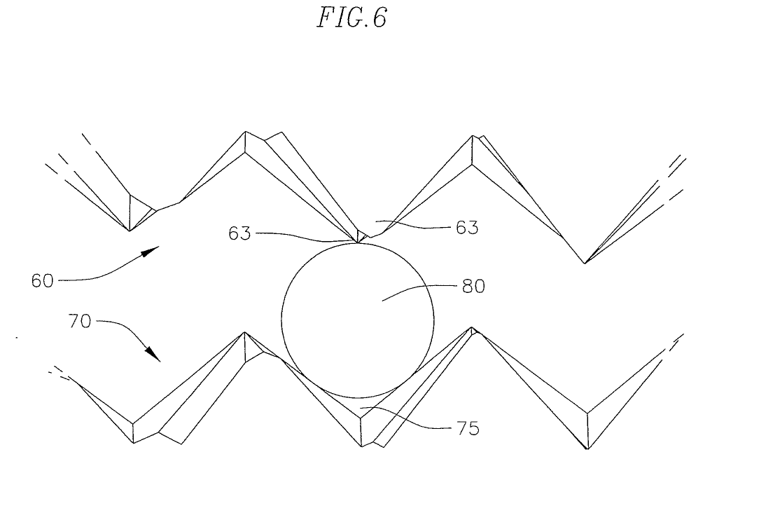

With regard to FIG. 6, several of the plurality of octagonal pyramidal teeth 70 forming a lower channel 75 and several of the upper apexes 63 engage a straight needle 80 in an orientation diagonal to the longitudinal axis of the needle driver 100. In an alternative orientation, not shown, several of the plurality of octagonal pyramidal teeth 60 forming an upper channel 65 and several of the lower apexes 73 engage a straight needle 80 in an orientation diagonal to the longitudinal axis of the needle driver 100.

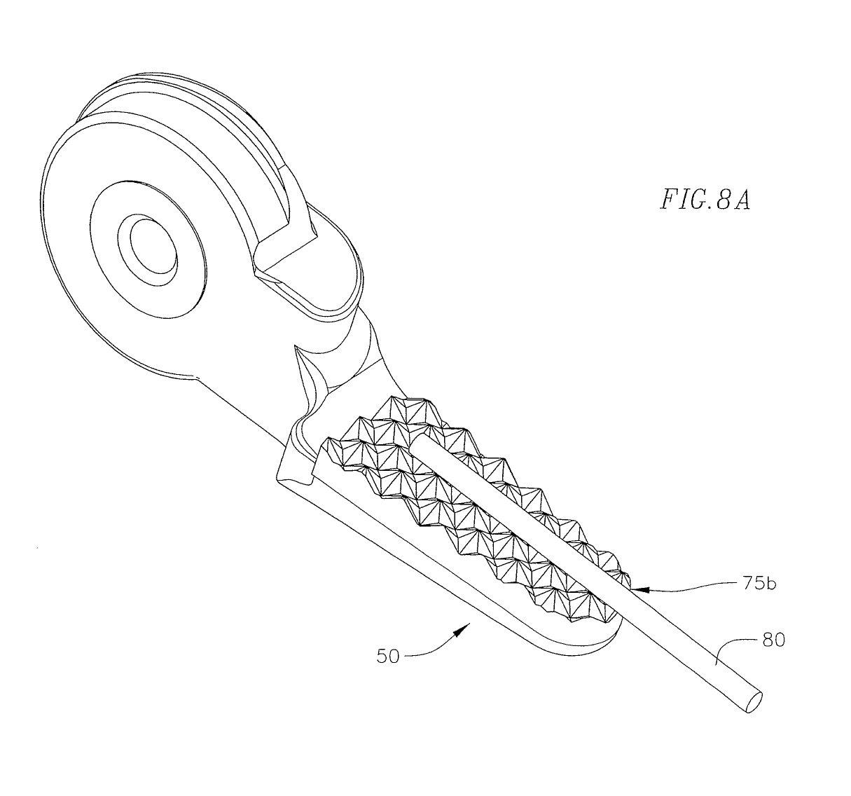

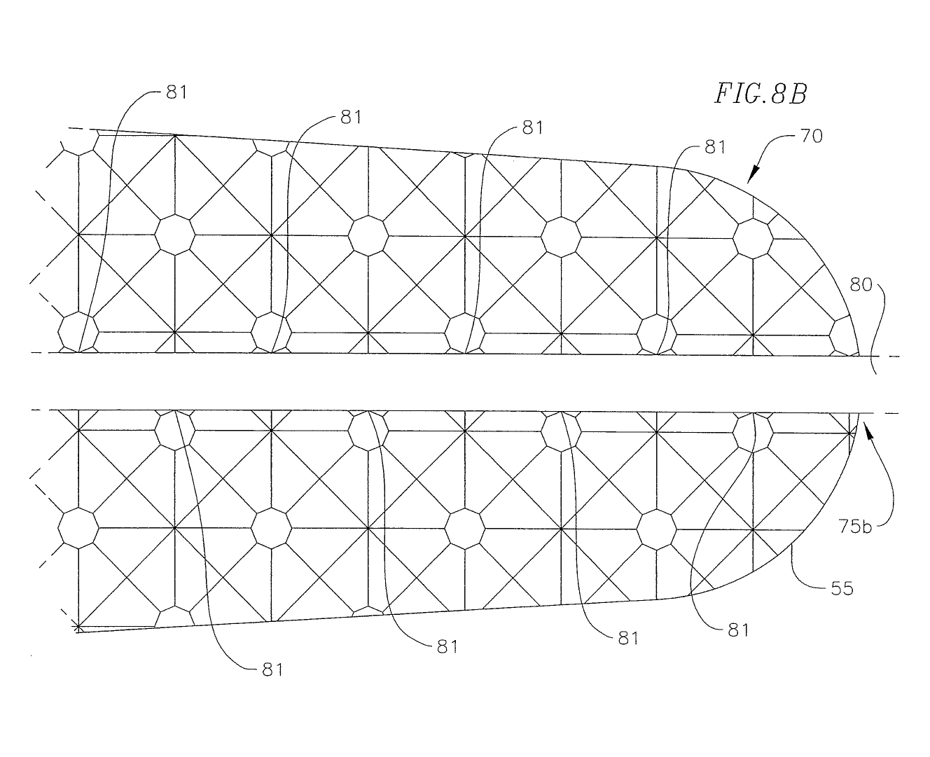

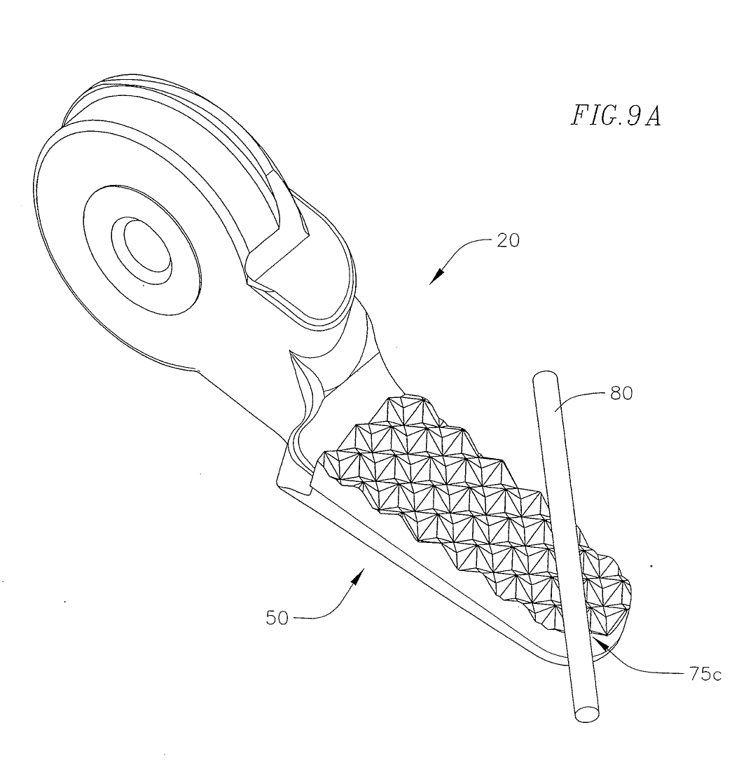

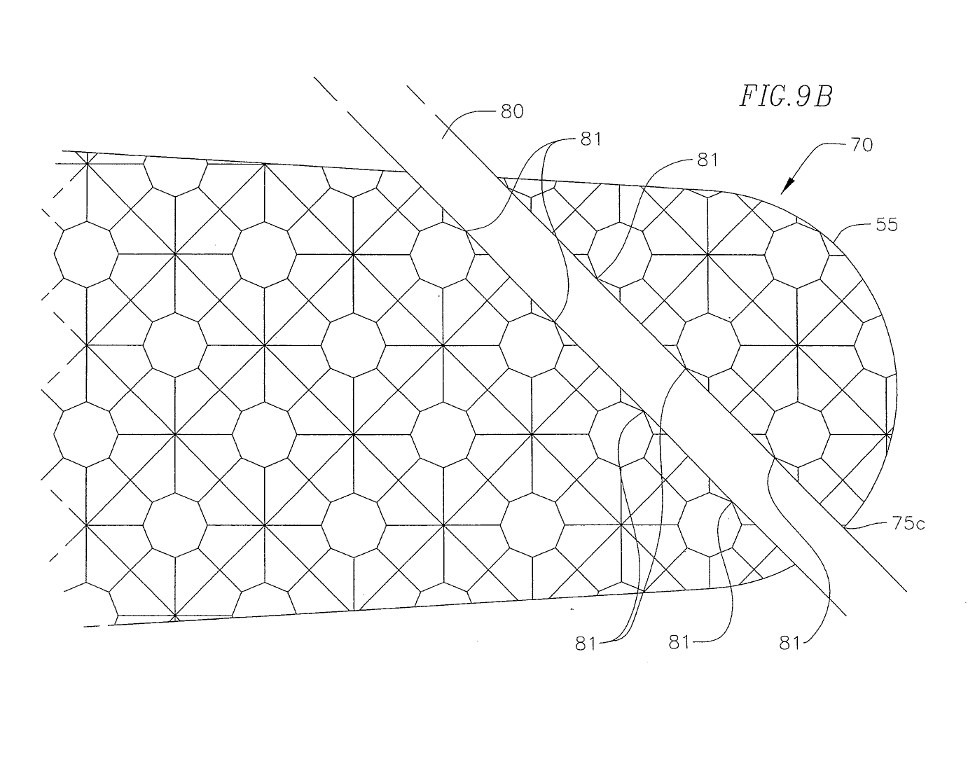

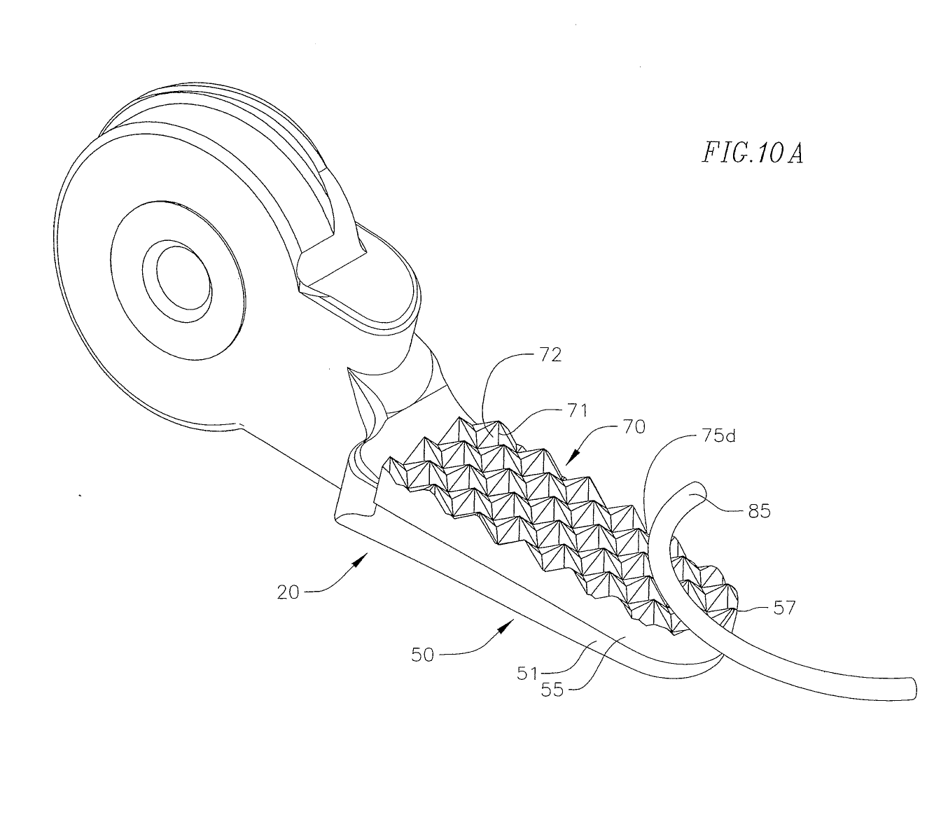

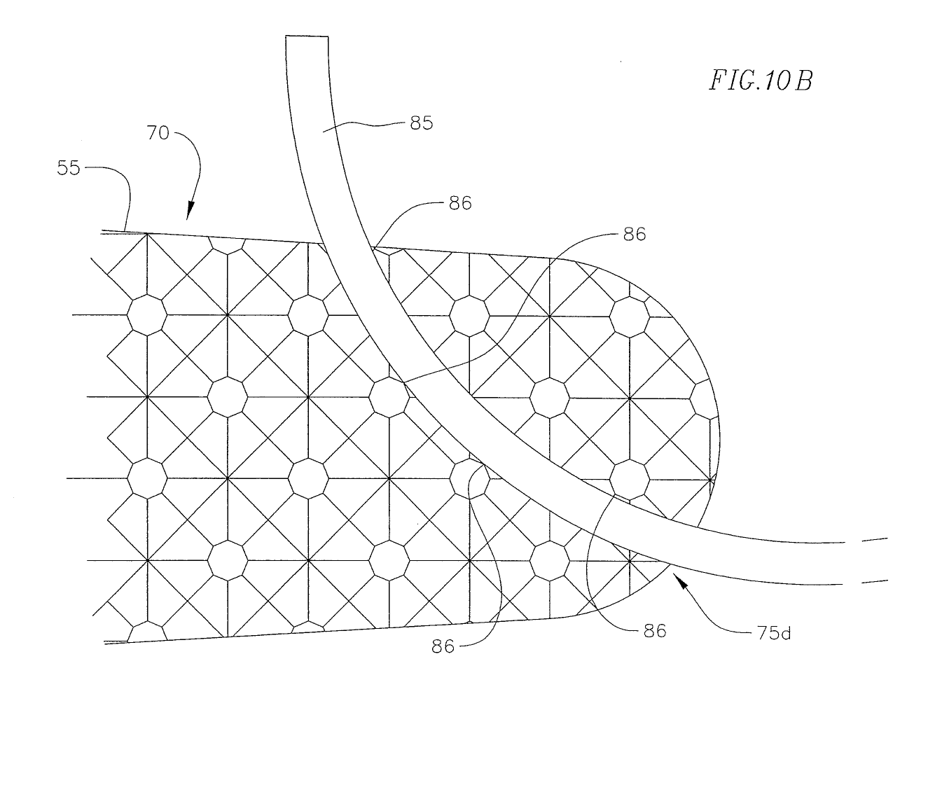

With regard to FIG. 7A, an embodiment of the present invention includes a second leg 20 comprising a lower jaw 50 with a plurality of lower octagonal pyramidal teeth 70. Each of the plurality of lower octagonal pyramidal teeth 70 has eight exposed edges 71 and eight exposed faces 72. A lower channel comprises a plurality of edges 71 and a plurality of faces 72 of several of the plurality of lower octagonal pyramidal teeth 70. Lower channels for engaging a straight needle in several orientations are recognized, for example 75a in a transverse orientation, 75b in a lengthwise orientation shown in FIGS. 8A and 75c in a diagonal orientation shown in FIG. 9A. Similarly, lower channels for engaging a curved needle in several orientations are also recognized for a curved needle, for example 75d shown in FIG. 10A.

With regard to FIG. 7A, a straight needle 80 is engaged in the lower channel 75a in a transverse orientation to the longitudinal axis of the lower jaw 50.

With regard to FIG. 7B, a cross-sectional view of the embodiment of the needle driver engaging a straight needle 80 as shown in FIG. 7A shows the plurality of points of contact 81 between the straight needle 80 and the edges of the several teeth of the plurality of lower octagonal pyramidal teeth 70 that comprise the lower channel 75a on the lower insert portion 55. It will be appreciated that depending on the diameter of the needle, the needle will rest higher or deeper in the channel. In one embodiment, the needle diameter is less than 1.3 times the peak-to-peak spacing between adjacent teeth. If the needle diameter is larger, the edges would no longer engage the needle, and only the apex of each tooth would engage the needle.

With regard to FIG. 8A, a straight needle 80 is engaged in the lower channel 75b in a lengthwise orientation to the longitudinal axis of the lower jaw 50.

With regard to FIG. 8B, a cross-sectional view of the embodiment of the needle driver engaging a straight needle 80 as shown in FIG. 8A shows the plurality of points of contact 81 between the straight needle 80 and the edges of the several teeth of the plurality of lower octagonal pyramidal teeth 70 that comprise the lower channel 75b on the lower insert portion 55.

With regard to FIG. 9A, a straight needle 80 is engaged in the lower channel 75c in a diagonal orientation to the longitudinal axis of the lower jaw 50.

With regard to FIG. 9B, a cross-sectional view of the embodiment of the needle driver engaging a straight needle 80 as shown in FIG. 9A shows the plurality of points of contact 81 between the straight needle 80 and the edges of the several teeth of the plurality of lower octagonal pyramidal teeth 70 that comprise the lower channel 75c on the lower insert portion 55.

With regard to FIG. 10A, a curved needle 85 is engaged in the lower channel 75d.

With regard to FIG. 10B, a cross-sectional view of the embodiment of the needle driver engaging a curved needle 85 as shown in FIG. 10A shows the plurality of points of contact 86 between the curved needle 85 and the edges of the several teeth of the plurality of lower octagonal pyramidal teeth 70 that comprise the lower channel 75d on the lower insert portion 55.

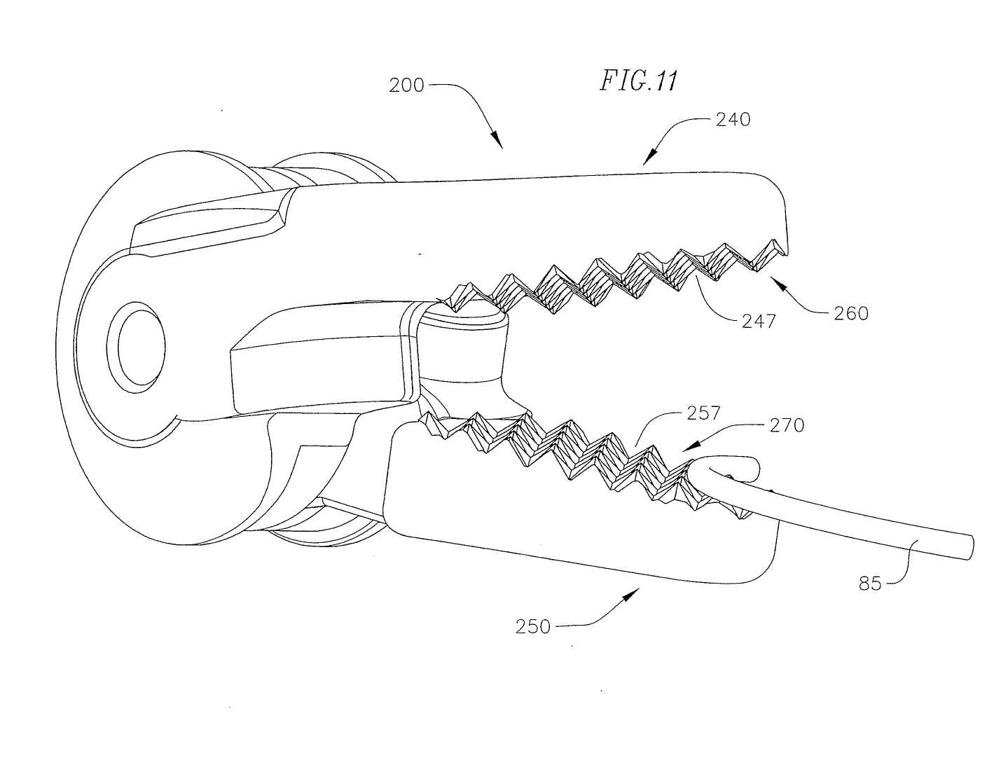

With regard to FIG. 11, an embodiment of a needle driver 200 comprises an upper jaw 240 and a lower jaw 250, and is shown engaging a curved needle 85. A plurality of upper octagonal pyramidal teeth 260 are situated on an upper outer surface 247 of the upper jaw 240. A plurality of lower octagonal pyramidal teeth 270 are situated on a lower outer surface 257 of a lower jaw 250. The upper jaw 240 comprises a single piece. In other words, the plurality of upper octagonal pyramidal teeth 40 and the upper jaw 240 are formed in a single piece. This upper jaw 240 may be referred to as a one-piece upper jaw 240. Similarly, the lower jaw 250 is formed in a single piece and may be referred to as a one-piece lower jaw 250. The one-piece upper jaw 240 and the one-piece lower jaw 250 together form a needle driver with a one-piece jaw 200.

In one embodiment, the needle driver 200 has a structure substantially similar to the one described above in reference to needle driver 100. A difference between the needle driver 100 and the needle driver 200 is the structure of the upper jaw and the lower jaw. In the needle driver 100, the upper jaw 40 and the lower jaw 50 are each comprised of a holder portion 41 and 51, respectively, and an insert portion 45 and 55, respectively. In the needle driver 200, the upper jaw 240 and the lower jaw 250 are each formed in a single piece. Other features of the needle driver 200, including the spacing and positioning of the pluralities of polygonal pyramidal teeth 260 and 270, the handle and the fulcrum, are substantially similar to those of the needle driver 100. Thus, a more detailed description of other parts of the needle driver 200 will not be provided.

With regard to FIG. 12, in an embodiment, a polygonal pyramidal tooth 90 of the pluralities of polygonal pyramidal teeth 60, 70, 260 or 270 has a plurality of exposed edges 91 and a plurality of exposed faces 92. A polygonal pyramidal tooth 90 having eight exposed edges 91 and eight exposed faces 92 is an octagonal pyramidal tooth 90.

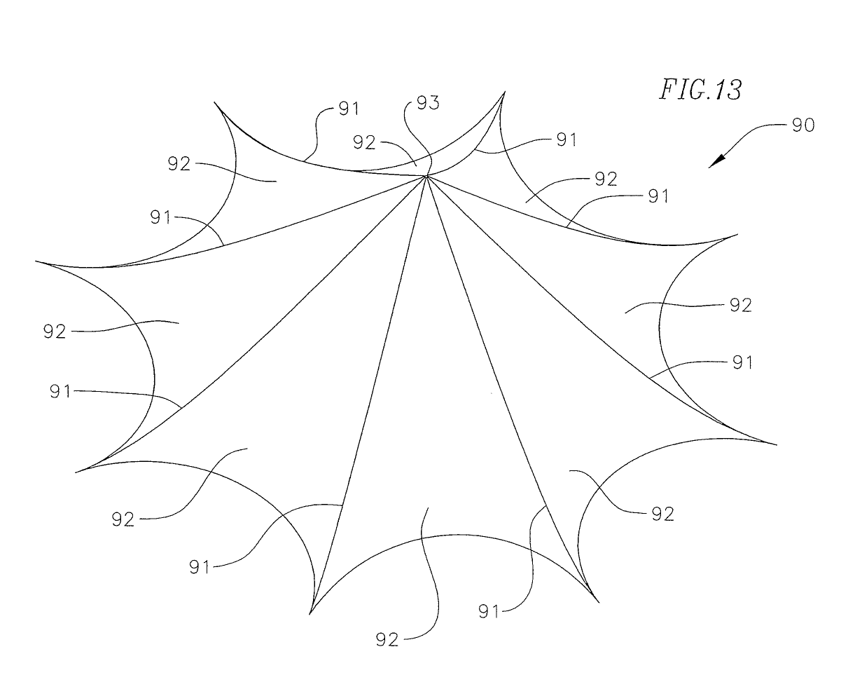

In one embodiment, shown in FIG. 12, the plurality of exposed faces 92 are planar. In other embodiments, the plurality of exposed faces 92 may be non planar. For example, as shown in FIG. 13, the plurality of exposed faces 92 in one embodiment are concave. A polygonal pyramidal tooth 90 with a plurality of concave exposed faces 92 has the feature that each of the plurality of edges 91 between each of the plurality of exposed faces 92 has a more acute angle than each of a plurality of exposed edges 91 between each of a plurality of planar exposed faces 92. An edge having a more acute angle has a smaller surface area contacting the needle. In turn, a smaller surface area concentrates a given clamping force for the needle driver. A sufficient clamping force per unit area will micro-dent the needle and provide a more secure grip. In contrast, a needle driver having four-sided polygonal pyramidal teeth that engages the needle with the faces of the teeth on the lower jaw can only micro-dent the needle with the apexes of the teeth on the upper jaw, which results in a less secure grip on the needle.

Further modifications of the embodiments described are possible. For example, each polygonal pyramidal tooth 90 may have greater than or fewer than eight exposed sides. However, since the exposed edges 91 engage the needle, a polygonal pyramidal tooth 90 with more than four exposed sides allows for a greater number of needle orientations with respect to the needle driver 100 or 200.

In one embodiment, each polygonal pyramidal tooth 90 has a pointed apex 93. In another embodiment, each polygonal pyramidal tooth 90 has an apex that is flat (not shown). In yet another embodiment, each polygonal pyramidal tooth 90 has an apex that is rounded (not shown).

The needle driver 100 or 200 may be made from any suitable material. Suitable materials for the first leg 10 and the second leg 20, and the analogous parts in the needle driver 200, include suitably strong and rigid materials suitable for use in a surgical setting. Suitable materials for the first leg 10 and the second leg 20 include stainless steel, but are not limited thereto.

The pluralities of polygonal pyramidal teeth 60, 70, 260 and 270 may be made from any suitably strong and hard material, including but not limited to carbide stainless steels, tool steels and intermetallics.

The needle driver 100 or 200 may be made by any suitable process, for example machining, casting or molding. The pluralities of polygonal pyramidal teeth 60, 70, 260 and 270; upper and lower jaws 40, 50, 240 and 25; upper and lower holding portions 41 and 51; and upper and lower insert portions 45 and 55 may each be made by any suitable process, including but not limited to machining, casting, pressing or powder injection molding.

Any of the components of the needle driver 100 or the needle driver 200 may be made separately from any of the other components. Any of the components of the needle driver 100 or the needle driver 200 may be made by a process that is different from the process used to make any of the other components.

Machining a plurality of polygonal pyramidal teeth from a suitably strong and hard material can be expensive and time consuming. Powder injection molding is known for making products for a broad range of applications, including automotive, aerospace, medical, dental and firearms. It has the feature of being able to precisely form a complex geometry, such as that of the polygonal pyramidal teeth, at a more economical cost and at a much faster rate than machining. A powder injection molding process can precisely produce complex features more efficiently than machining or conventional casting.

In a preferred embodiment of the present invention, an entire jaw of the needle driver or component parts of the needle driver are made from a powder injection molding process. Such a process includes mixing a powder of either metal or ceramic with a polymer binding powder to form a feed stock; injecting the feed stock into a mold suitably sized and shaped to produce the desired component; and cooling the molded part, also called a green part, so it hardens. Optionally, the green part may be machined. The powder injection molding process also includes removing some of the polymer binder from the green part in a process called debinding, which may include a catalytic chemical, solvent or water debind process; and sintering the resulting part, also called a brown part, by heating it to about 400 to 800° C. to remove residual polymer binder and to about 1200 to 2200° C. to refine the material properties of the metal or ceramic part. The sintering may be accomplished by any suitable process, for example heating in a vacuum-type furnace. Optional steps after the sintering may include further modifying the part, for example by machining, heat treating, coating, finishing or coining; and/or increasing the density of the sintered part by a hot isostatic pressing step. The specific parameters of a powder injection molding process vary depending on the material used, the geometry of the desired component and the characteristics desired. Such variations are known to those skilled in the art, thus a more detailed description will not be provided.

Powder injection molding has an additional feature of allowing the entire needle driver 200, or at least the one-piece upper jaw 240 with a plurality of upper polygonal pyramidal teeth 260 or the one-piece lower jaw 250 with a plurality of lower polygonal pyramidal teeth 270, to be molded in a single piece of suitable strong and hard material, including but not limited to carbide, stainless steels, tool steels or intermetallics. Thus, the one-piece upper jaw 260 or the one-piece lower jaw 270 has teeth integrated with the jaw itself, and the teeth are sufficiently hard and strong to achieve a desired performance of the needle driver 200. This feature may offer the one-piece upper jaw 240 or the one-piece lower jaw 250 different structural characteristics from an upper jaw 40 with an upper insert portion 45 or a lower jaw 50 with a lower insert portion 55, since there would be no joint or seam interrupting the one-piece upper jaw 240 or lower jaw 250. There could also be economic efficiencies in eliminating a joining step in a manufacturing process of the upper jaw 40 or the lower jaw 50 comprising a holder portion 41 and 51, respectively, and an insert portion 45 and 55, respectively.

1-11. (canceled)

12. A method of manufacturing a surgical needle driver comprising:
powder injection molding at least one surgical needle driver component selected from a group consisting of a jaw, a jaw holder portion, a jaw insert portion and a plurality of polygonal pyramidal teeth, wherein each of the plurality of polygonal pyramidal teeth has more than four faces;
sintering the at least one surgical needle driver component at an elevated temperature to form at least one sintered surgical needle driver component; and
assembling the at least one sintered surgical needle driver component with the other surgical needle driver components such that the surgical needle driver comprises an upper jaw with an outer surface having a plurality of polygonal pyramidal teeth and a lower jaw with an outer surface having a plurality of polygonal pyramidal teeth.

13. The method of claim 12, wherein powder injection molding the at least one surgical needle driver component comprises preparing a mold for the at least one surgical needle driver component.

14. The method of claim 12, wherein powder injection molding the at least one surgical needle driver component comprises powder injection molding a one-piece jaw including a plurality of polygonal pyramidal teeth.

15. The method of claim 12, wherein powder injection molding the at least one surgical needle driver component comprises powder injection molding a one-piece leg including a jaw.

16. The method of claim 12, wherein the at least one sintered surgical needle driver component comprises at least one selected from carbide, stainless steels, tool steels, technical ceramics, or intermetallics.

17. A method of manufacturing a surgical needle driver comprising the steps of:
blending a powder and a binder to form a powdered feedstock composition;
providing a mold having a mold cavity defining a monolithic surgical needle driver component;
injecting said powdered feedstock composition into said mold to form a green monolithic surgical needle driver component;
debinding said green component such that said binder separates from said powder to form a powdered surgical needle driver component;
and sintering said powdered surgical needle driver component at an elevated temperature to form a sintered monolithic surgical needle driver component.

18. The method of claim 17, wherein said powder is a powder selected from carbide, stainless steels, tool steels, technical ceramics, or intermetallics, and said binder is a polymer binder.

19. The method of claim 17, further comprising machining said green component.

20. The method of claim 17, wherein the method comprises heating said green monolithic surgical needle driver component to a range of 400 to 800° C. to remove a residual portion of the binder.

21. The method of claim 17, wherein debinding said green monolithic surgical needle driver component comprises leaching said green monolithic surgical needle driver component in a solvent to remove a residual portion of the binder.

22. The method of claim 17, wherein the method comprises heating the powdered surgical needle driver component to a range of 1200 to 2200° C. to consolidate the powder and refine material properties of the powdered surgical needle driver component.

23. The method of claim 17, further comprising modifying the sintered monolithic surgical needle driver component by at least one of machining, heat treating, coating, finishing, coining, or hot isostatic pressing.
